(19) United States
(12) Patent Application Publication
(10) Pub. No.: US 2008/0269688 A1
(43) Pub. Date: Oct. 30, 2008

(54) Dose Indicating Assembly of a Pharmaceutical Injection Device

(76) Inventors: Jose Colucci, Lexington, MA (US); Elizabeth Whitney Johansen, Somerville, MA (US); Timothy Lee Moulton, Boston, MA (US); Charles Richard Okenfuss, Carmel, IN (US)

Correspondence Address: ELI LILLY & COMPANY, PATENT DIVISION, P.O. BOX 6288, INDIANAPOLIS, IN, 46206-6288, US

(21) Appl. No.: 12/095,038

(22) PCT Filed: Dec. 5, 2006
(86) PCT No.: PCT/US06/61600

§ 371 Date: May 27, 2008

Related U.S. Application Data
(60) Provisional application No. 60/748,636, filed on Dec. 8, 2005.

(51) Int. Cl. A61M 5/31

(52) U.S. Cl. 604/189

(57) ABSTRACT

A dose indicating assembly in a pharmaceutical injection device (20). The dose indicating assembly includes an external housing barrel (31) extending in an axial direction, and a dial at least partially disposed within the housing barrel. The dial (33) is screwably movable in the axial direction relative to the barrel during dose setting. The dial includes an outer radial periphery with a plurality of parallel arrays (38, 39, 40) of dose indicia provided thereon, each of the plurality of arrays of dose indicia provided in a helical pattern on the periphery. The dose indicating assembly also includes means (50) for viewing the dose indicia of a selectively chosen one of the plurality of arrays.

### BACKGROUND OF THE INVENTION

The present invention pertains to pharmaceutical injection devices, and, in particular, to an assembly that indicates a dose the device is set to administer.

Patients suffering from a number of different diseases frequently must inject themselves with pharmaceuticals. A variety of delivery devices such as injection pens have been developed to facilitate such injections. Injection pens typically allow a user to set a dose to administer, and then to further operate the pen to inject that set dose.

At least one injectable medication, namely human growth hormone, is available with different cartridges containing different concentrations of the medication. As for a given volume of delivery such different cartridges result in the administration of different amounts of the medication, systems have been developed to help people use these cartridges to conveniently administer an intended amount of medication. One known system uses color coding of the cartridges, where there is a color-coded corresponding injection pen that is graduated to deliver the appropriate dose based on the concentration of medicine in the cartridge. While helpful, the need to supply the market with different pens increases complexity and may raise costs. Another proposed system uses an electronic pen, which pen senses a concentration marking of the cartridge, and uses that sensed marking to calculate and then display the actual dose of medication to be administered based on the volume the pen has been prepared to deliver. While this system advantageously allows a single pen to be used with different concentration cartridges, the cartridge recognition function of such pen may be undesirably expensive or complicated to achieve.

Thus, it would be desirable to provide a system that can overcome one or more of these and other shortcomings of the prior art.

### BRIEF SUMMARY OF THE INVENTION

In one form thereof, the present invention provides a dose indicating assembly, in a pharmaceutical injection device, including an external housing barrel extending in an axial direction, a dial at least partially disposed within the housing barrel, the dial screwably movable in the axial direction relative to the barrel during dose setting, the dial including an outer radial periphery with a plurality of parallel arrays of dose indicia provided thereon, each of the plurality of arrays of dose indicia provided in a helical pattern on the periphery, and means for viewing the dose indicia of a selectively chosen one of the plurality of arrays.

One advantage of the present invention is that a single type of injection device can be used conveniently with different concentration cartridges.

Another advantage of the present invention is that an injection device can be converted easily and quickly, such as by a medical professional, for proper use of its contents selected for loading.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a perspective, diagrammatic view of a pharmaceutical injection device equipped with one embodiment of a dose indicating assembly of the present invention;

FIG. 2 is a partial, elevational view of the dial removed from the remainder of the pharmaceutical injection device of FIG. 1, wherein the dose indicia arrays of the dose indicating assembly are further shown;

FIG. 3 is a two-dimensional representation of the dose indicia arrays that wrap around the circumference of the dial of FIG. 2;

FIGS. 4 and 5 are two-dimensional representations of different, alternate dose indicia arrays of the dial;

FIG. 6 is a perspective, diagrammatic view of a pharmaceutical injection device equipped with another embodiment of a dose indicating assembly of the present invention;

FIG. 7 is a perspective, diagrammatic view of a pharmaceutical injection device equipped with still another embodiment of a dose indicating assembly of the present invention;

FIG. 8 shows a series of selectable labels of the dose indicating assembly of FIG. 7;

FIG. 9 is a perspective, diagrammatic view of a pharmaceutical injection device equipped with another embodiment of a dose indicating assembly of the present invention;

FIG. 10 is a two-dimensional representation of the dose indicia arrays that wrap around the circumference of the dial in the pharmaceutical injection device of FIG. 9;

FIGS. 11, 12 and 13 are two-dimensional representations of different, alternate dose indicia arrays for a dial for the device of FIG. 9;

FIG. 14 is a partial perspective, diagrammatic view of a pharmaceutical injection device equipped with another embodiment of a dose indicating assembly of the present invention;

FIG. 15 is a partial perspective, diagrammatic view of a pharmaceutical injection device equipped with another embodiment of a dose indicating assembly of the present invention; and

FIG. 16 is an exploded, perspective view of portions of a pharmaceutical injection device equipped with another embodiment of a dose indicating assembly of the present invention.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

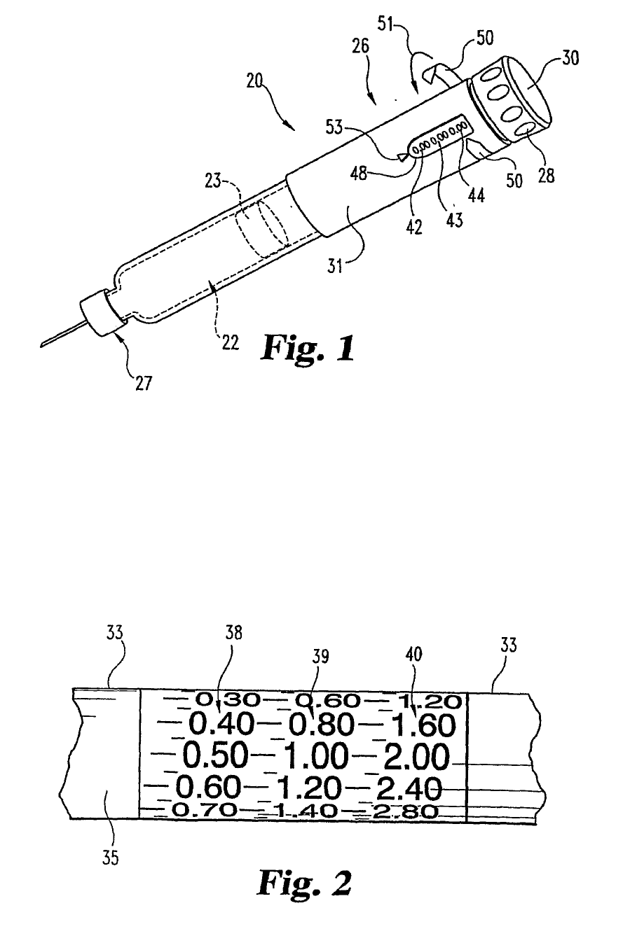

FIG. 1 diagrammatically illustrates one type of pharmaceutical injection device equipped with a dose indicating assembly of the present invention. The shown device is an injection pen, generally designated 20. As with many known models of injection pens, injection pen 20 includes a standard medication-filled cartridge 22 having a movable piston 23. The cartridge 22 is operatively assembled to a base portion, generally designated 26, of pen 20. For example, cartridge 22 may be an integral part of a cartridge assembly that is connected to the pen base, or may be separately inserted and held within a retainer that is then connected to the pen base.

Pen base 26 includes dose setting and injecting mechanisms that function to allow a quantity of medicine to be selected and then expelled from cartridge 22 through the injection needle assembly 27 shown mounted to the device in fluid communication with the cartridge contents. The specifics of the dose setting and injecting mechanisms of injection pen 20 are only briefly described herein as such are not material to the present invention, other than the operational relationship to the dose-indicia displaying dial.

In the embodiment of FIG. 1, a knob 28 with an independently rotatable button 30 thereon is exposed at the rearward or proximal end of pen base 26. Knob 28 and button 30 are manually operable portions of the dose setting and injecting mechanisms otherwise housed within the cylindrical housing barrel 31 of pen base 26. Knob 28 is operatively connected with a cylindrical, tubular dial 33 that includes dose indicia and which fits within barrel 31 when device 20 is arranged in a ready-to-set state shown in FIG. 1. During the dose setting process, knob 28 is designed to be rotated relative to the housing barrel 31 to set the dose, and when knob 28 is so rotated to increase the selected dose the knob 28 and dial 33 screw out from barrel 31 from the axial position shown in FIG. 1, or to the upper right from the perspective of a FIG. 1 viewer. During the dose injecting process initiated by the user after the dose setting process, as the drive button 30 is plunged forward, the knob 28 and dial 33 screw back toward the barrel 31, with the dial region that had been exposed beyond the barrel proximal end during dose setting being returned within the barrel, and with such plunging resulting in the injecting mechanism of the device causing the set dose of medicine to be delivered from the cartridge 22 through the needle assembly 27.

The foregoing device description is provided as background and is intended to be illustrative and not limiting in any way, as, for example, a variety of known injection devices may find beneficial application of the dose indicating assembly of the present invention. In particular, the inventive dose indicating assembly may be adapted for injection devices of different shapes, sizes and configurations having a dose indicating dial that moves during dose setting, and typically dose injecting as well. For example, the housing barrel need not be cylindrical, but may be more box-shaped. And, the inventive dose indicating assembly is applicable to autoinjectors, and further does not require the presence of a dose setting mechanism that allows variability in the volume to be delivered.

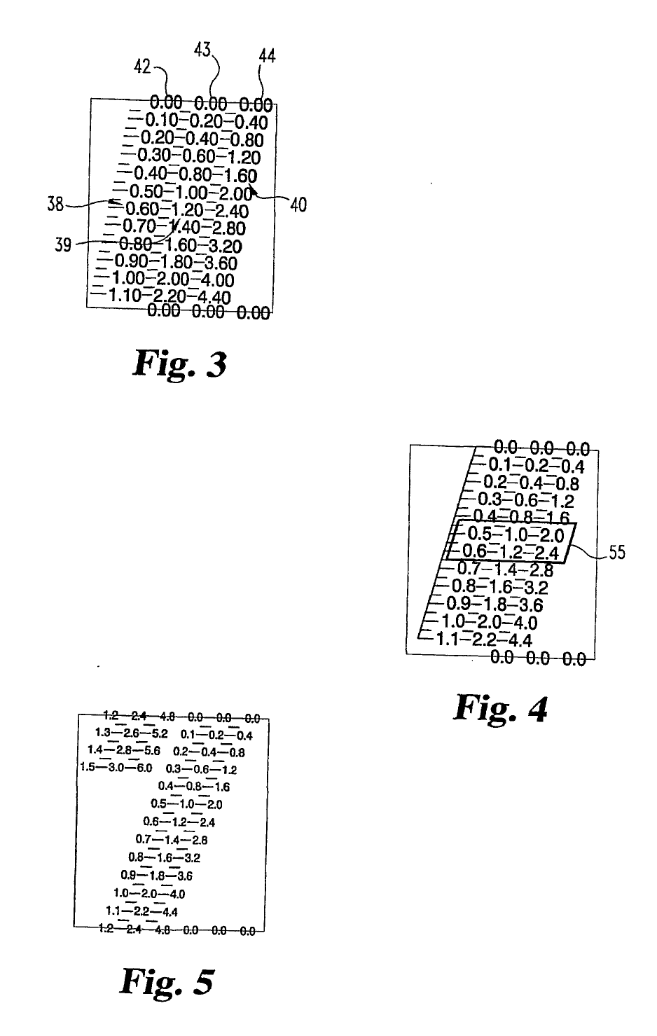

With additional reference to FIG. 2 and FIG. 3, dial 33 of the dose indicating assembly includes an outer radial periphery 35 with a plurality of arrays of dose indicia provided thereon. The plurality of arrays is shown as a first array, a second array and a third array, designated 38, 39 and 40, respectively. Other numbers of arrays, including as few as two, may be employed within the scope of the invention. Three arrays is suitable for one form of human growth hormone available in three milliliter cartridges each containing one of six, twelve or eighteen milligrams of lyophilized drug, with the concentrations of the cartridge contents after reconstitution reflecting these weights.

Each of arrays 38-40 is provided in a spiraling or helical pattern, relative to the axial direction, on the dial periphery. The helical patterns are shown as being parallel, being coaxial with a common pitch and diameter, and further share or overlap a common axial segment of the dial length without the digits of the various patterns covering each other. Each of dose indicia arrays 38, 39 and 40 includes a zero dose marking, namely 42, 43 and 44, respectively. In the stacked arrangement shown, zero dose markings 42-44 not only are spaced from each other in an axial direction, but also are axially aligned, such that the opening in the barrel through which the dose indicia are to be visible may be axially oriented.

The viewing mechanism for the dose arrays of FIGS. 2 and 3 is provided in the form of an aperture 48 allowing visibility of each of arrays 38-40. Aperture 48 is formed as a hole in the barrel periphery which is completely ringed by that barrel, but could be provided in another form. A marker is provided, such as in the form of a sticker strip 50 that is adhesively securable to the pen base barrel 31. Marker strip 50 is selectively applied, such as shown at 51, by a health care professional or other pen preparer, including possibly the patient user or possibly the manufacturer, to point to the one of arrays 38-40 that corresponds to the concentration of medicine which is contained within the loaded cartridge. Strip 50 is sized and configured to wrap around nearly the entire barrel circumference with its pointed ends adjacent aperture 48.

An arrow 53 or other focusing feature, as is known, is shown adjacent the distal axial end of aperture 48 on the barrel periphery to point to a row of indicia, especially when the aperture 48 spans a portion of the barrel circumference large enough to allow multiple numbers or markings, corresponding to different dose amounts, of each helical array to be visible. For example, the outline indicated at 55 in FIG. 4 represents one suitable aperture design in which multiple numbers of each helical array are simultaneously visible, and arrow 53 would serve to direct the user to the proper number of the helical array pointed to be sticker 50.

The heights and widths of the digits used in the helical arrays naturally may be modified by one skilled in the art. For example, FIGS. 4 and 5 show other possible helical dose indicia arrays that may be provided with suitable modifications to the configuration of the internal workings of the injection device. The arrays of the inventive dose indicating assembly may be provided in various manners known in the art, such as printing directly on the dial periphery, or a preprinted label sized to exactly wrap around the complete dial circumference as is illustrated in FIGS. 2-5.

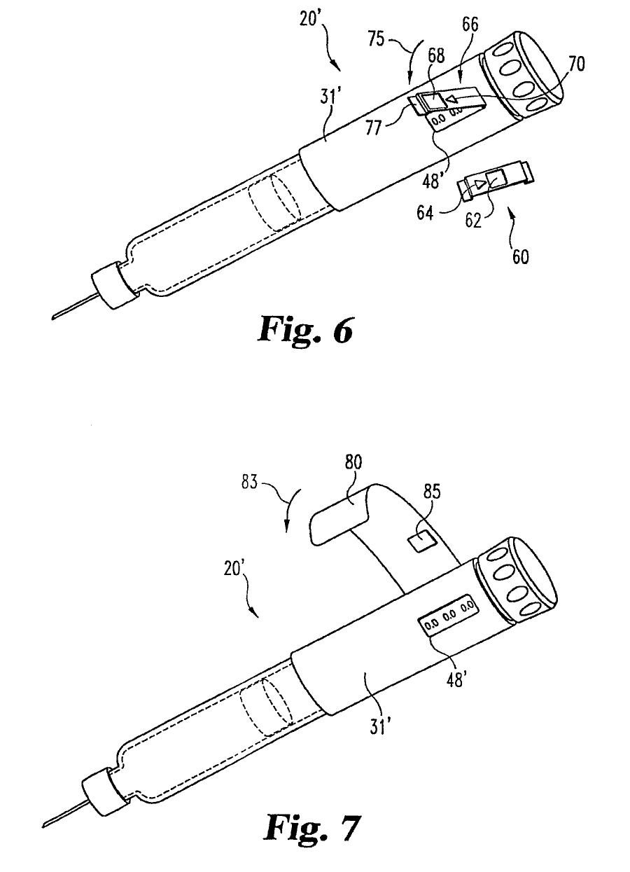

Referring now to FIG. 6, there is shown an alternate embodiment of the present invention in injection pen 20′. The dial indicia may be similar to that disclosed with respect to the embodiment of FIG. 1, in that each of the dose indicia arrays is at least initially simultaneously visible through a single, axially oriented aperture 48′ in barrel 31′. The indicating assembly includes two snap inserts. Insert 60 is intended to be used to reveal the middle of the three arrays, while covering or obscuring the other two arrays, and includes a window 62 therethrough which is centered along the axial length of the insert. Arrow 64 is arranged at the center of the window. Insert 66 is intended to reveal either of the arrays that flank the middle array, and cover the other two arrays, and includes a window 68 positioned axially off-center along the axial length of the insert. Arrow 70 is arranged at the center of the window. Inserts 60 and 66 are individually, and mutually exclusively, snap fit to barrel 31′ over aperture 48′ to obscure two of the three helical arrays, leaving or revealing only the appropriate concentration information through its respective window. Insert 66 is shown in FIG. 6 about to be bent in at 75 and snap fit via its tabs 77 to barrel 31′ to expose the array closest to the needle end of device 20′, but alternatively may be rotated 180 degrees and snap fit into aperture 48′ to expose the array farthest from the needle end of device 20′. Snap inserts 60 and 66 may be installed, for example, by the health care professional, who could switch inserts, or reorient the installed insert, first by inserting the tip of a paper clip or dedicated tool behind the window and prying off the insert, and then installing the proper, or properly re-oriented, insert.

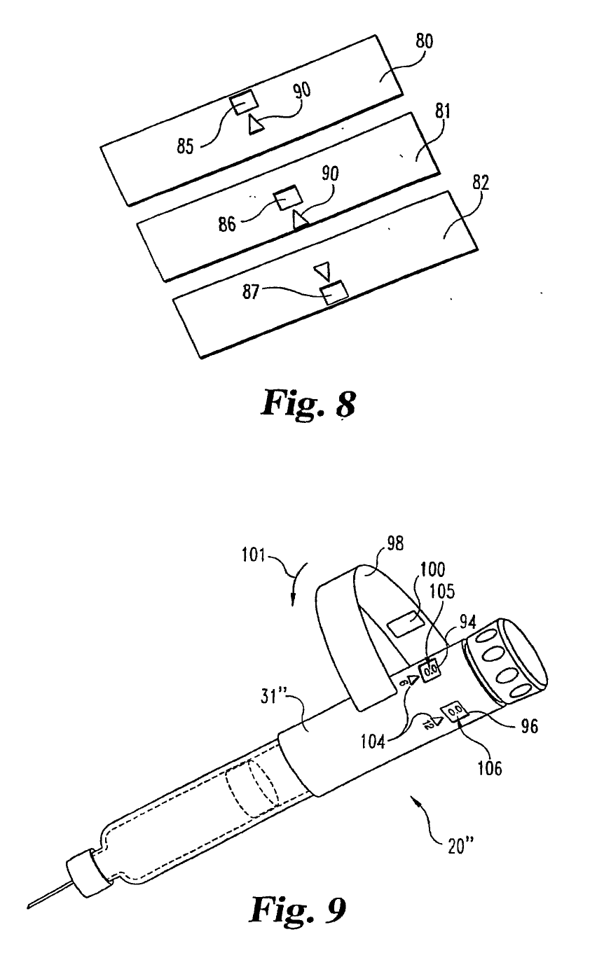

Referring now to FIGS. 7 and 8, there is shown still another embodiment of the present invention in an injection pen the same as pen 20′. Barrel 31′ includes a single window 48′, but the device comes with two or three different markers, such as the three markers in the form of adhesive strips 80, 81 and 82 shown separate from the remainder of pen 20′ in FIG. 8. Only one of strips 80-82 is used. Marker 81 includes a centered hole or window 86, while markers 80 and 82 each have a have window, 85 and 87, respectively, provided on a non-centered portion. Each marker may be provided with other information helpful to a pen preparer and/or user, such as an arrow 90 or a notice as to the concentration of medicine with which that particular marker is intended to be utilized. Each marker, when applied as being performed with strip 80 as indicated at 83, covers aperture 48′ such that only one array is visible.

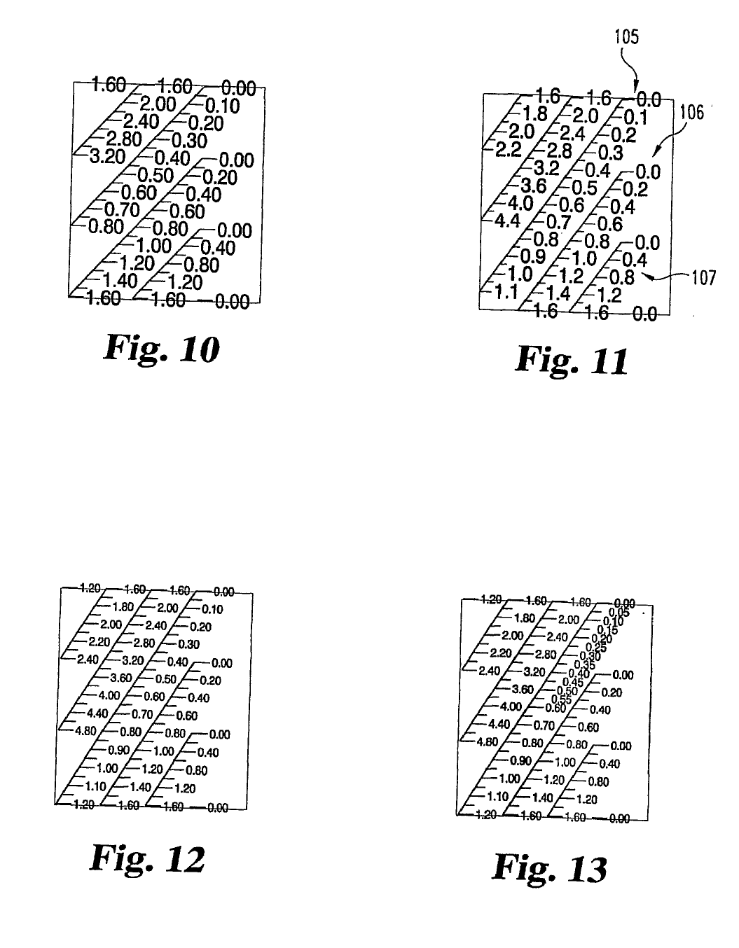

Referring now to FIG. 9, there is shown still another embodiment of the present invention in an injection pen 20″. In this embodiment, the plurality of parallel arrays of dose indicia on the dial are arranged such that their zero dose markings are angularly or circumferentially spaced on the dial, and more particularly are disposed at the same axial position along the length of the dial. Two or more arrays may be employed within the scope of the invention. FIGS. 10 through 13 illustrate, in two-dimensional format, various types of designs having three arrays that may be employed within the scope of the invention. For purposes of further describing the embodiment of FIG. 9, the arrays 105, 106 and 107 of FIG. 11 will be referenced.

In FIG. 9, the indicating assembly allows for viewing of the arrays 105-107 through a plurality of apertures formed through the barrel, and through each of which a different dose indicia array is visible. More particular, a first aperture 94, a second aperture 96, and a third aperture (not shown) allow visibility only of arrays 105, 106 and 107, respectively. These three apertures are equally spaced around the circumference of barrel 31″ at an even axial location. A marker in the form of an adhesive label or strip 98 is provided with an aperture 100, which marker strip can be wrapped around the barrel exterior by, for example, a health care provider as shown at 101 to leave uncovered only the barrel aperture and its associated dose indicia array appropriate for the concentration of the medication contained in the device. Numerals are shown at 104 next to centering arrows which may provide further indication for, for example, a doctor as to which concentration that aperture and array are associated, which numbers may or may not be obscured by the label.

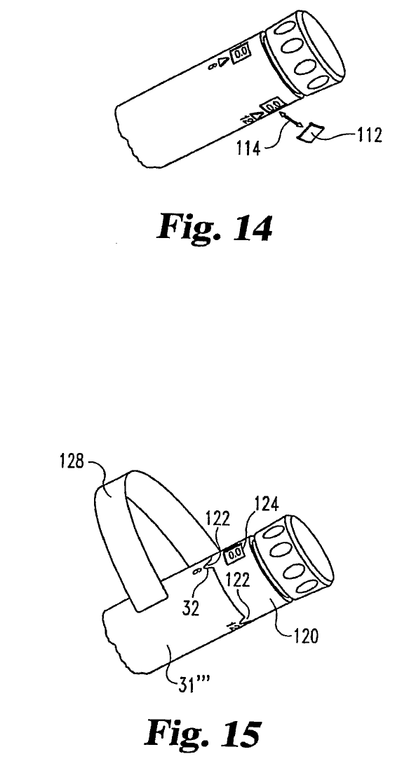

Referring now to FIG. 14, there is shown another alternate embodiment of the invention used with injection pen 20″. Two identical inserts 112 may be snap inserted as shown at 114 by, for example, the health care provider into two of the three equally angularly spaced apertures to leave a single aperture uncovered, which single uncovered aperture exposes only the appropriate concentration information. In a not shown, related alternate embodiment, rather than two separate snap inserts that fit over two of three apertures, the barrel may be provided with the apertures being identifiable, but either already all covered with inserts, or otherwise provided with readily removable, such as scored, portions of the barrel itself, one of which may be selectively removed to correspond to the appropriate concentration.

Referring now to FIG. 15, there shown another embodiment of the present invention which includes a dose indicia displaying dial similar to that of the embodiment of FIG. 9. The housing barrel 31″′ is provided with a keyed proximal end 32, which keying is adapted to be used with an opaque, ring-shaped collar 120 of the indicating assembly which has three equally angularly spaced keys 122 that mate with the barrel keying at any of three angular orientations. Collar 120 includes a single aperture 124 aligned axially with one of keys 122. The pen preparer, such as the health care provider, is able to rotate collar 120 to align keys 122 with the keying in order for aperture 124 to be angularly located such that only the appropriate concentration information is visible through collar aperture 124. Numerals shown at 125 corresponding to concentrations can help the provider with the collar alignment. When the proper angular orientation of collar 120 relative to barrel 31″′ is provided, a label 128 may be secured around their abutting interface to axially and rotatably fix together the barrel and the collar.

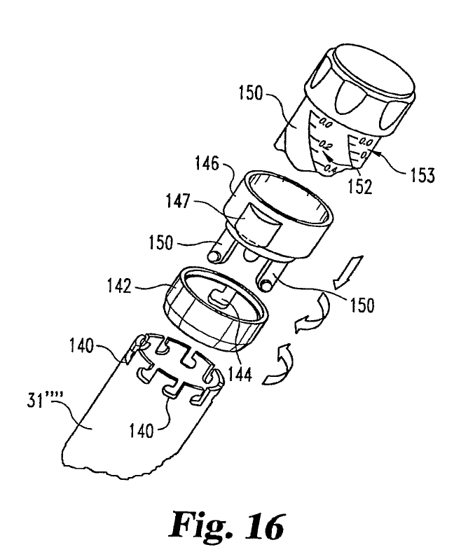

Referring now to FIG. 16, there is abstractly shown another embodiment of the present invention. The dial 150 that screws out from and into barrel 31″″ has three parallel helical arrays, two of which are shown at 152 and 153. The array digits are circumferentially oriented, as opposed to the axial orientation shown with the other embodiments. Barrel 31″″ includes a proximal end including a series of bayonet slots 140. A locking ring. 142, with a radially protruding tab 144 fits around barrel 31″″. The indicating assembly also includes a ring-shaped collar 146 with a lens covered window 147, such as a magnifying lens. Three depending ears 150 interfit with slots 140 to allow collar 146 to be selectively mounted to the barrel such that the appropriate one of the dose indicia arrays is visible through window 147. After so mounting the collar to the barrel, locking ring 142 may be rotated to lock the collar to the barrel

All of the foregoing allow a health care provider or other pen preparer to adapt an injection pen to a concentration of the medicine contained therein. It will be appreciated that the pen may be a reusable device in that, for example, the health care provider may replace spent cartridges with similar replacement cartridges for reuse. Alternatively, especially in the case of the embodiments of FIG. 16 or those that utilize replaceable inserts, the pen, if reusable, may be modified for delivery of different concentrations of medicines.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

We claim:

1. In a pharmaceutical injection device, a dose indicating assembly comprising:
an external housing barrel extending in an axial direction;
a dial at least partially disposed within the housing barrel, said dial screwably movable in the axial direction relative to the barrel during dose setting, said dial including an outer radial periphery with a plurality of parallel arrays of dose indicia provided thereon, each of said plurality of arrays of dose indicia provided in a helical pattern on said periphery; and
means for viewing the dose indicia of a selectively chosen one of said plurality of arrays.

2. The dose indicating assembly of claim 1 wherein each of said plurality of arrays of dose indicia includes a zero dose marking, and wherein said zero dose markings are spaced in an axial direction on said dial.

3. The dose indicating assembly of claim 2 wherein said zero dose markings are axially aligned.

4. The dose indicating assembly of claim 1 wherein each of said plurality of arrays of dose indicia includes a zero dose marking, and wherein said zero dose markings are angularly spaced on said dial.

5. The dose indicating assembly of claim 4 wherein said angularly spaced zero dose markings are disposed at the same axial position on said dial.

6. The dose indicating assembly of claim 1 wherein said plurality of arrays of dose indicia comprises a first array, a second array, and a third array.

7. The dose indicating assembly of claim 1 wherein said viewing means comprises an aperture formed in a periphery of said barrel for visibility of said arrays of dose indicia, and at least one insert with a window mountable within said aperture such that the dose indicia of a selected array is visible through the insert window.

8. The dose indicating assembly of claim 7 wherein said at least one insert comprises first and second inserts for mutually exclusive mounting within said aperture, said first insert and said second insert each having a window differently located along their respective axial lengths.

9. The dose indicating assembly of claim 1 wherein said viewing means comprises at least one aperture formed in a periphery of said barrel for visibility of said arrays of dose indicia, and at least one marker selectably securable to said housing.

10. The dose indicating assembly of claim 9 wherein said at least one aperture comprises a plurality of separate apertures, and said at least one marker is adapted to obscure visibility through all but one of said plurality of apertures.

11. The dose indicating assembly of claim 9 wherein said at least one aperture comprises a single aperture, and said at least one marker comprises a single marker that points to dose indicia of a selected array.

12. The dose indicating assembly of claim 9 wherein said at least one aperture comprises a single aperture, and said at least one marker comprises a set of markers each with a window differently axially positioned so as to allow visibility of dose indicia of different arrays when individually secured.

13. The dose indicating assembly of claim 1 wherein said viewing means comprises a ring with an aperture for dose indicia visibility, and means for securing said ring to said barrel at a selected angular orientation allowing visibility of dose indicia of a selected array through said aperture.

14. The dose indicating assembly of claim 13 wherein said securing means comprises a keyed fit between said barrel and said ring.

15. The dose indicating assembly of claim 14 wherein said securing means further comprises a rotatable locking ring.

16. The dose indicating assembly of claim 14 wherein said securing means further comprises an adhesive strip interconnecting said ring and said barrel.

17. The dose indicating assembly of claim 1 wherein at least a portion of said helical arrays occupy a common axial segment of said dial.
